(19) United States
(12) Patent Application Publication
(10) Pub. No.: US 2023/0420138 A1
(43) Pub. Date: Dec. 28, 2023

(54) DEVELOPING ADAPTABLE PREDICTIVE ANALYTICS FOR SUBJECTS IN MEDICAL FACILITIES

(71) Applicant: KONINKLIJKE PHILIPS N.V., EINDHOVEN (NL)

(72) Inventors: SAMAN PARVANEH, Danvers, MA (US); BRYAN CONROY, Garden City South, NY (US)

(21) Appl. No.: 18/033,393

(22) PCT Filed: Oct. 18, 2021
(86) PCT No.: PCT/EP2021/078733

§ 371 Date: Apr. 24, 2023

Related U.S. Application Data
(60) Provisional application No. 63/104,630, filed on Oct. 23, 2020.

(51) Int. Cl. G16H 50/30; G16H 50/20; G16H 40/20; A61B 5/00

(52) U.S. Cl. CPC G16H 50/30; G16H 50/20; G16H 40/20; A61B 5/7282; A61B 5/743

(57) ABSTRACT

A method is provided for developing adaptable predictive analytics for subjects in a medical facility. The method includes training a predictive algorithm (S311) for predicting an adverse medical event at a desired notification time before the medical event using initial annotations indicating times for diagnosis of the medical event; determining real risk scores over time (S312) using the predictive algorithm, where the real risk scores indicate actual probabilities of the medical event occurring at predetermined times before the medical event; creating required risk scores (S313) based on a real risk score trend, where the required risk scores indicate modified probabilities of the medical event occurring at the predetermined times; mapping the initial annotations to a time-series of new annotations (S314) that minimizes differences between the required and real risk scores; fine-tuning the predictive algorithm (S315) using the time-series of new annotations; and monitoring subjects (S316) by applying the fine-tuned predictive algorithm.

### BACKGROUND

There has been a steady increase in the amount of data collected through a variety of inputs from patients in hospitals and other clinical environments, especially intensive care units (ICUs). Predictive analytics for predicting various adverse medical events in patients, such as hemodynamics instability (HI), stroke, atrial fibrillation prediction, acute kidney injury (AKI), pressure injury (PI), respiratory distress, acute lung injury, and risk of infection, for example, have been developed to assist with patient management using the collected data. Generally, predictive analytics predict adverse medical events in patients in medical facilities using the collected data, enabling clinicians to observe predictive scores over time and make timely decisions on patient management in response. The data are annotated to enable the development and use of predictive analytics.

However, the annotations used for creating predictive algorithms have limited availability, often provided for only one instance, or very few instances, during each patient's stay in the medical facility. Using available annotations for predicting an adverse medical event may not meet clinical requirements, such as policies in prevention and optimum use of resources. For example, too early of a prediction of an adverse medical event may be considered a false positive under the clinical requirements or policies. Also, an early prediction of the adverse medical event may result in a premature response under the clinical requirements, which is problematic when there are insufficient resources or may result in overstaffing to address adverse medical events that are not imminent.

Accordingly, reliable and adaptable predictive analytics are needed, where the prediction of the adverse medical events may be adjusted to suit requirements of different medical facilities, as desired. Such adaptable predictive analytics would fully benefit from a limited number of annotations during a patient stay and meet the requirements specific to each medical facility.

### SUMMARY

According to an aspect of the present disclosure, a method of developing adaptable predictive analytics for subjects in a medical facility is provided. The method includes training a predictive algorithm or other artificial intelligence (AI) model of the predictive analytics for predicting an adverse medical event at a desired notification time before occurrence of the adverse medical event using initial annotations indicating times for diagnosis of the adverse medical event, and determining real risk scores over time using the predictive algorithm applied to the subjects in the medical facility, and identifying a corresponding trend of the real risk scores, where the real risk scores indicate actual probabilities of the adverse medical event occurring at predetermined times before the occurrence of the adverse medical event. The method further includes creating required risk scores over time based on the real risk score trend, where the required risk scores indicate modified probabilities of the adverse medical event occurring at the predetermined times before the occurrence of the adverse medical event; mapping the initial annotations to a time-series of new annotations that minimizes differences between the required risk scores and the real risk scores; and fine-tuning the predictive algorithm for predicting the adverse medical event at the desired notification time using the time-series of new annotations. At least one of the subjects in the medical facility is monitored by applying the fine-tuned predictive algorithm to indicate the desired notification time.

According to another aspect of the present disclosure, a system is provided for developing adaptable predictive analytics for subjects in a medical facility. The system includes an interface for receiving initial annotations indicating times for diagnosis of an adverse medical event in subjects of the medical facility; a processor in communication with the interface; and a memory in communication with the processor. The memory stores instructions that, when executed by the processor, causes the processor to perform a method including training a predictive algorithm for predicting the adverse medical event at a desired notification time before occurrence of the adverse medical event using the initial annotations; determining real risk scores over time using the predictive algorithm applied to the subjects in the medical facility, and identifying a corresponding trend of the real risk scores, where the real risk scores indicate actual probabilities of the adverse medical event occurring at predetermined times before the occurrence of the adverse medical event; creating required risk scores over time based on the real risk score trend, where the required risk scores indicate modified probabilities of the adverse medical event occurring at the predetermined times before the occurrence of the adverse medical event; mapping the initial annotations to a time-series of new annotations that minimizes differences between the required risk scores and the real risk scores; fine-tuning the predictive algorithm for predicting the adverse medical event at the desired notification time using the time-series of new annotations; and monitoring at least one of the subjects in the medical facility by applying the fine-tuned predictive algorithm to indicate the desired notification time.

According to another aspect of the present disclosure, a non-transitory computer readable medium that stores instructions that, when executed by a computer processor, performs a method for developing adaptable predictive analytics for subjects in a medical facility. The method is performed by executing steps including receiving initial annotations indicating times for diagnosis of an adverse medical event in subjects of the medical facility; training a predictive algorithm for predicting the adverse medical event at a desired notification time before occurrence of the adverse medical event using the initial annotations; determining real risk scores over time using the predictive algorithm applied to the subjects in the medical facility, and identifying a corresponding trend of the real risk scores, where the real risk scores indicate actual probabilities of the adverse medical event occurring at predetermined times before the occurrence of the adverse medical event; creating required risk scores over time based on the real risk score trend, where the required risk scores indicate modified probabilities of the adverse medical event occurring at the predetermined times before the occurrence of the adverse medical event; mapping the initial annotations to a time-series of new annotations that minimizes differences between the required risk scores and the real risk scores; and fine-tuning the predictive algorithm for predicting the adverse medical event at the desired notification time using the time-series of new annotations, and for monitoring at least one of the subjects in the medical facility by applying the fine-tuned predictive algorithm to indicate the desired notification time.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a graph showing examples of trends of hemodynamics instability (HI) scores for stable and unstable subjects used for predicting HI occurrence in subjects.

FIG. 2 is a simplified block diagram of a system for developing adaptable predictive analytics for subjects in a medical facility, according to a representative embodiment.

FIG. 3 is a simplified flow diagram of a method for developing adaptable predictive analytics for subjects in a medical facility, according to a representative embodiment.

FIG. 4A is a graph showing an example of a real risk score trend over time, according to a representative embodiment.

FIG. 4B is a graph showing an example of a required risk score trend over time, corresponding to the real risk score trend of FIG. 4A, according to a representative embodiment.

FIG. 5 is a simplified block diagram of an artificial intelligence (AI) engine for developing adaptable predictive analytics for subjects in a medical facility, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

According to various embodiments, automated determination and development of adaptable predictive analytics for predicting occurrence of an adverse medical event for subjects (e.g., patients) in medical facilities are provided using limited initial annotations of data collected from subjects in the medical facilities over time. An “adverse medical event” is a discrete or acute manifestation of a medical condition experienced by a subject (e.g., patient), the occurrence of which is predictable based on various physiological biomarkers and/or symptoms known to precede the manifestation. As mentioned above, examples of adverse medical events include, but are not limited to, HI, stroke, atrial fibrillation prediction, AKI, PI, respiratory distress, acute lung injury, and risk of infection. A “medical facility” is a clinical establishment organized to provide medical treatment to subjects (e.g., patients) in accordance with pre-established policies. Examples of medical facilities include, but are not limited to, hospitals, emergency care clinics, rehabilitation centers, and nursing homes.

The initial annotations may indicate times for diagnosis of the adverse medical event for the subjects specific to the medical facility. The adaptable predictive analytics include a predictive algorithm that is developed and tuned using artificial intelligence (AI) based on occurrence of the adverse medical event using the initial annotations, and that indicates a desired notification time before occurrence of the adverse medical event (e.g., one hour before occurrence of the adverse medical event). The predictive algorithm may be an AI model, such as a recurrent neural network (RNN) based model, for example. Generally, a set of training data relating to a particular adverse medical event is created for pre-training the predictive algorithm, for example, using data and/or initial annotations of data, which may be collected over time from subjects in the medical facility and/or from subjects in other similar medical facilities providing similar care. The training data set may be used to populate a database (e.g., database 220). Feature values of desired features associated with various relevant physiological characteristics are extracted from the training data set, and the extracted feature values are applied to the predictive algorithm to determine respective contributions of the features to the predicted occurrence of the adverse medical event. An example of developing and training a predictive algorithm, which relates to prediction of hemodynamic instability (HI) risk for a subject, is described by U.S. Patent Application No. 2019/0029533 (published Jan. 31, 2019) to Potes et al., which is hereby incorporated by reference in its entirety. Predictive algorithms relating to other types of adverse medical events may be developed and pre-trained in substantially the same manner using different corresponding data and/or data annotations, and features, as would be apparent to one skilled in the art. The desired notification time is the amount of time before an anticipated adverse medical event that the medical facility had determined to be an appropriate amount of time to properly prepare for the eventuality of the adverse medical event. The desired notification time may be established, and revised as needed, by the policies of the medical facility, for example.

Using trends of predictive scores over time (real risk scores), feedback is collected by the medical facility to create adjusted predictive scores over time (required risk scores) that effectively isolate the desired notification time before occurrence of the adverse medical event, preventing early, undesired notification. Using the required risk scores, a time-series of new annotations may be created based on the initial annotations (usually one or limited number of annotations), and the new annotations will be used for retraining, e.g., fine-tuning, the predictive algorithm and/or for creating a new predictive algorithm. This improves patient management by the medical facility, while maintaining awareness of pending adverse medical events that require medical attention, in a cost effective manner.

Generally, predictive analytics for healthcare applications, designed to predict adverse medical events at a specific time before the adverse medical events (e.g., one hour), are also able to predict the adverse medical events at even earlier times (e.g., three hours). For example, annotations related to hemodynamic instability (HI) may be available for a specific time instance, e.g., time zero (the time of occurrence of HI), and a trained model may be formed according to the predictive analytics for the purpose of predicting occurrence of the HI one hour before the specific time instance. However, the trained model may also generate results that predict the HI earlier than one hour before the specific time instance, which may cause problems associated with early prediction, discussed above.

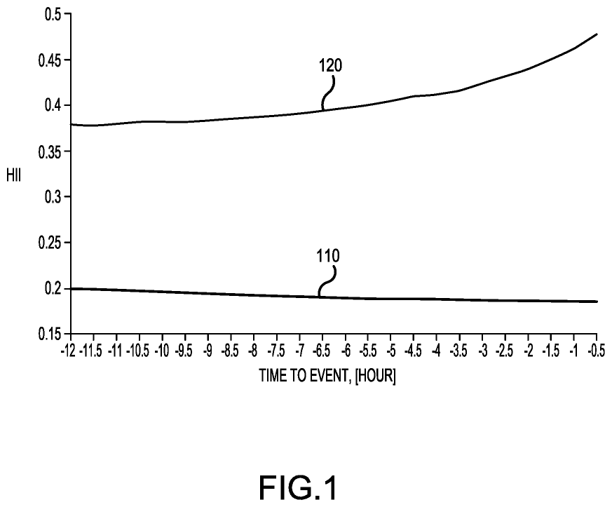

In this regard, FIG. 1 is a graph showing examples of trends of HI scores (HI index) for stable and unstable subjects used for predicting HI occurrence in subjects according to predictive analytics. In particular, trace 110 shows a trend of HI scores for stable subjects and trace 120 shows a trend of HI scores for unstable subjects, who are more susceptible to HI. The HI scores are plotted as a function of time, marked in half hour increments, before occurrence of HI at time zero. Traces 110 and 120 are based on data from the eICU Collaborative Research Database. See T. J. Pollard et al., “The eICU Collaborative Research Database, a freely available multi-center database for critical care research,” Scientific Data (2018).

Trace 110 shows a steady, relatively low risk of HI. Trace 120 shows a higher risk of HI throughout the relevant time period, wherein the HI index increases from about 0.38 at 12 hours before time zero to about 0.47 at 0.5 hour before time zero, where time zero is the time of the HI event. At about three hours before time zero, trace 120 shows an increased rate at which the HI index increases. This indicates that the predictive algorithm may be used to predict HI three hours before the adverse medical event, even though it was intended to predict only one hour before the adverse medical event. Such an early prediction of increased risk score may be perceived as a false positive, or may be unacceptable by the medical facility due to internal policies or lack of resources to respond to early predictions. Therefore, according to various embodiments, adaptable predictive patient analytics are provided in order to adjust the time of prediction, enabling the medical facility to benefit fully from the limited number of annotations and also to meet the internal requirements, as discussed below.

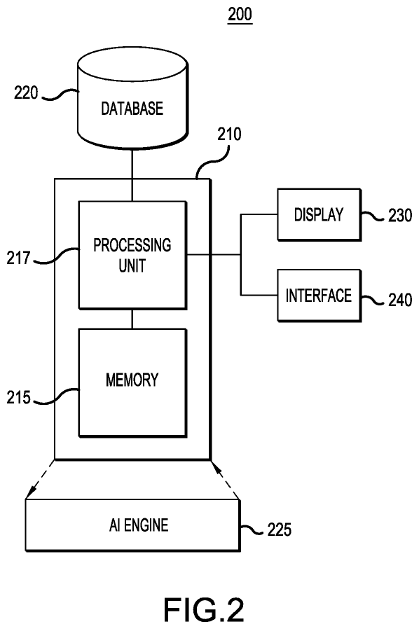

FIG. 2 is a simplified block diagram of a system for developing adaptable predictive analytics for subjects in a medical facility, according to a representative embodiment.

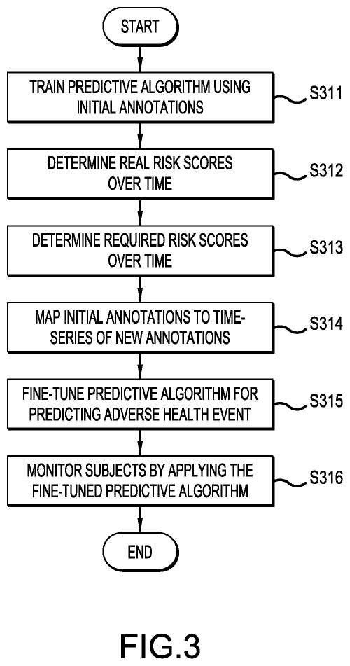

Referring to FIG. 2, computer system 200 includes a computer 210, a database 220, an AI engine 225, a display 230 and an interface 240. The computer 210 in the computer system 200 includes a memory 215 that stores instructions and a processing unit 217 that executes the instructions. When executed, the instructions cause the processing unit 217 to implement a process that includes developing adaptable predictive analytics, an example of which is shown in FIG. 3. In addition, the processing unit 217 may implement additional operations based on executing instructions, such as instructing or otherwise communicating with another element of the computer system 200, including the database 220, the display 230 and the interface 240, to perform one or more of the above-noted processes.

The processing unit 217 is representative of one or more processing devices, and is configured to execute software instructions to perform functions as described in the various embodiments herein. The processing unit 217 may be implemented by field programmable gate arrays (FPGAs), application specific integrated circuits (ASICs), a general purpose computer, a central processing unit, a computer processor, a microprocessor, a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The memory 215 may include a main memory and/or a static memory, where such memories may communicate with each other and the processing unit 217 via one or more buses. The memory 215 stores instructions used to implement some or all aspects of methods and processes described herein. The memory 215 may be implemented by any number, type and combination of random access memory (RAM) and read-only memory (ROM), for example, and may store various types of information, such as software algorithms, AI models including RNN and other neural network based models, and computer programs, all of which are executable by the processing unit 217. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, blu-ray disk, a universal serial bus (USB) drive, or any other form of storage medium known in the art. The memory 215 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 215 may store software instructions and/or computer readable code that enables performance of various functions. The memory 215 may be secure and/or encrypted, or unsecure and/or unencrypted.

Similarly, the database 220 stores data and instructions used to implement some or all aspects of methods and processes described herein. The database 2220 may be implemented by any number, type and combination of RAM and ROM, for example, and may store various types of information, such as software algorithms, AI models including RNN and other neural network based models, and computer programs, all of which are executable by the processing unit 217. The various types of ROM and RAM may include any number, type and combination of computer readable storage media, such as a disk drive, flash memory, EPROM, EEPROM, registers, a hard disk, a removable disk, tape, CD-ROM, DVD, floppy disk, blu-ray disk, USB drive, or any other form of storage medium known in the art. The database 220 is a tangible storage medium for storing data and executable software instructions and are non-transitory during the time software instructions are stored therein. The database 220 may be secure and/or encrypted, or unsecure and/or unencrypted.

“Memory” and “database” are examples of computer-readable storage media, and should be interpreted as possibly being multiple memories or databases. The memory or database may for instance be multiple memories or databases local to the computer, and/or distributed amongst multiple computer systems or computing devices.

The AI engine 225 may be implemented as software that provides artificial intelligence (e.g., a predictive algorithm) and applies machine learning described herein. Although depicted separately in FIG. 2, it is understood that the AI engine 225 may reside in any of various components, such as the computer 210, the database 220, a patient monitor, a server, and/or the cloud, for example. The AI engine 225 may be connected to the computer 210 via a local wired interface such as an Ethernet cable or via a local wireless interface such as a Wi-Fi connection, or may be remote from the computer 210, connected via the internet using one or more wired connection(s) and/or wireless connection(s). As mentioned above, the AI engine 225 may be implemented in a cloud, such as at a data center, for example, in which case the AI engine 225 may be connected to the computer 210 via the internet using one or more wired and/or wireless connection(s). The AI engine 225 may be connected to multiple different computers including the computer 210, so that the artificial intelligence and machine learning are performed centrally based on and for a relatively large set of medical facilities and corresponding subjects at different locations. Alternatively, the AI engine 225 may implement the artificial intelligence and the machine learning locally to the computer 210, such as at a single medical facility.

The interface 240 may one or more of include ports, disk drives, wireless antennas, or other types of receiver circuitry. For example, the computer 210 may retrieve or otherwise receive data and instructions via the interface 240 from a website, an email, a portable disk or other type of memory, including the database 220. The interface 240 may further connect one or more user interfaces, such as a mouse, a keyboard, a microphone, a video camera, a touchscreen display, voice or gesture recognition captured by a microphone or video camera, for example.

The display 230 may be a monitor such as a computer monitor, a television, a liquid crystal display (LCD), an organic light emitting diode (OLED), a flat panel display, a solid-state display, or a cathode ray tube (CRT) display, or an electronic whiteboard, for example. The display 230 may also include one or more interface(s), such as the interface 240, in which case the display 230 may provide a graphical user interface (GUI) for displaying and receiving information to and from a user.

FIG. 3 is a flow diagram showing a method of developing adaptable predictive analytics for subjects in a medical facility, according to a representative embodiment. The method may be implemented by computer readable instructions or computer code, e.g., stored the memory 215 and executable by the processing unit 217 of the computer 210, discussed above. More particularly, all or part of the method may be implemented by the AI engine 225, discussed above.

Referring to FIG. 3, the method includes training a predictive algorithm in block S311 for predicting an adverse medical event at a desired notification time before occurrence of the adverse medical event. The desired notification time may be entered via an interface (e.g., interface 240) and/or a graphical user interface by a user (e.g., clinician), and displayed on a display (e.g., display 230). The predictive algorithm is trained using initial annotations indicating times for diagnosis of the adverse medical event. The initial annotations are of data collected from subjects in the medical facility over time, and/or from subjects in other similar medical facilities, and may be retrieved from a database (e.g., database 220) and/or via an interface (e.g., interface 240), for example. The initial annotations are limited in that only one or a few annotations per subject are typically collected during each subject's stay in the medical facility.

The predictive algorithm itself may be any predictive algorithm known in the art for predicting an adverse medical event at a desired notification time before occurrence of the adverse medical event. The predictive algorithm may be implemented and trained by an AI engine, and the initial annotations may be collected and stored in a database accessible by the AI engine. For example, when the adverse medical event to be addressed is HI occurrence, data annotations may be collected over time regarding subjects in the medical facility who experience HI, including the actual time of the HI occurrence. Other data annotations may indicate the lengths of time the subjects are in the facility, preexisting conditions and medical condition of the subjects, and the severity of symptoms leading up to the HI occurrence, for example.

The desired notification time before occurrence of the adverse medical event to which the predictive algorithm is directed may vary based on the particular needs and policies of the medical facility using the predictive algorithm. Also, the desired notification time before occurrence of the adverse medical event may differ within the medical facility for different adverse medical events. For example, the medical facility may want a two hours notification time with respect to HI occurrence and a one hour notification time with respect to atrial fibrillation occurrence.

In an embodiment, the medical facility may alter the notification time with regard to a particular adverse medical event depending on immediate circumstances, such as staffing and other resource availability, as well as the policies of the medical facility. For example, when staffing is low compared to the number of subjects in the medical facility, the notification time may be lengthened (e.g., from two hours before the occurrence of the adverse medical event to three hours before) in order to give the staff additional advanced notice to anticipate and prepare for the adverse medical event. Or, when the staffing is low, the notification time may be shortened (e.g., from two hours before the occurrence of the adverse medical event to one hour before) to avoid having to address prematurely the adverse medical event in accordance with the medical facility's policies. That is, the earlier notification may produce a false positive that is difficult and disruptive to address when understaffed. Conversely, the notification time may be set early enough to enable the medical facility to adjust the available resources, as needed, to properly respond to the anticipated upcoming adverse medical events. For example, the medical facility may set the notification time to four hours before the adverse medical event to allow enough time for additional staff to be called in to handle an unexpectedly high number of these adverse medical events occurring at about the same time.

In block S312, real risk scores over time are determined using the predictive algorithm applied to the subjects in the medical facility and/or other similar medical facilities. For example, the real risk scores may be generated by applying the predictive algorithm to data collected from the subjects at various times relative to the adverse medical event. The real risk scores indicate actual probabilities of the adverse medical event occurring at predetermined times before the occurrence of the adverse medical event. The real risk scores collectively provide a corresponding trend of real risk scores, which effectively is a curve indicating the real risk scores as a function of time. The real risk scores may be determined for discrete times or time intervals (e.g., every ½ hour) over a predetermined period of time (e.g., 12 hours) before the predicted occurrence of the adverse medical event. Alternatively, the real risk scores may be determined as a continuum over the predetermined period of time before the occurrence of the adverse medical event.

In block S313, required risk scores over time are determined based on the real risk score trend provided by the real risk scores. The required risk scores indicate modified probabilities of the adverse medical event occurring at the predetermined times before the occurrence of the adverse medical event. Like the real risk scores, the required risk scores may be determined for discrete times or time intervals over the predetermined period before the predicted occurrence of the adverse medical event, or may be determined as a continuum over the predetermined period of time. The required risk scores collectively provide a corresponding trend of required risk scores, which effectively is a curve indicating the required risk scores as a function of time.

The modified probabilities reflect adjustments to the corresponding real risk scores to better isolate the desired notification time. This prevents predictive indications of the adverse medical event prior to the desired notification time. For example, in block S313, the values of the real risk scores may be adjusted to zero to provide the required risk scores at all predetermined times before the occurrence of the adverse medical event preceding the desired notification time. The required risk scores within the desired notification time reflect the same trend of the real risk scores within the desired notification time. So, for example, when the desired notification time is two hours before the adverse medical event, the required risk scores are zero for times occurring than earlier than two hours before the adverse medical event, and increase at substantially the same rate as the real risk scores for times occurring later than the two hours before the adverse medical event.

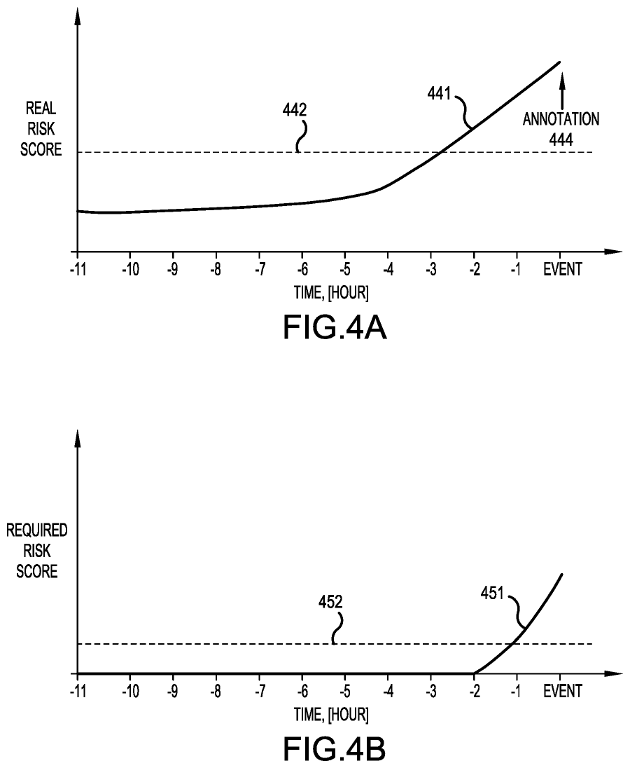

FIG. 4A is a graph showing an example of a real risk score trend over time, and FIG. 4B is a graph showing an example of the corresponding required risk score trend over time, according to a representative embodiment.

Referring to FIG. 4A, trace 441 indicates the real risk score trend determined over time using the predictive algorithm that predicts the desired notification time prior to occurrence of the adverse medical event. As discussed above, the real risk score trend may be determined using at least one initial annotation 444 that indicates the time for actual diagnosis of the adverse medical event. For purposes of illustration, it may be assumed that the predictive algorithm has been trained based on a desired notification time of two hours before the adverse medical event. As shown by trace 441, however, the real risk scores provided by the predictive algorithm begin to increase in value at about four hours before the adverse medical event. This means that the predictive algorithm may provide predictions of the adverse medical event at times up to two hours earlier than desired, which may result in inappropriate responses by the medical facility, as discussed above.

Referring to FIG. 4B, trace 451 indicates the required risk score trend determined using the real risk score trend based on the desired notification time. Because the desired notification time is two hours before the occurrence of the adverse medical event, the required risk score is set to zero for the times prior to two hours. Between the notification time of two hours and the time of occurrence of the adverse medical event, the slope of trace 451 is substantially the same as the slope of trace 441, indicating that the probability of the adverse medical event occurring increases with time at substantially the same rate determined using the real risk score trend (although at lower risk score values overall). Therefore, using the required risk score, the likelihood of occurrence of the adverse medical event is zero prior to two hours before the adverse medical event, and the likelihood of occurrence of the adverse medical event increases after two hours at a rate that substantially matches the rate provided by the real risk score trend. Accordingly, no prediction of the adverse medical event is possible prior to two hours, preventing early notification or alert, i.e., prior to the desired notification time. In an embodiment, a graph similar to trace 451 may be displayed for each subject on a display (e.g., display 230) in order to for track the desired notification time with respect to the subject.

In block S314, the initial annotations are mapped to a time-series of new annotations that minimizes differences between the required risk scores and the real risk scores. RNN may be used, for example, for mapping the data collected from the subjects to the initial annotations, as defined based on feedback from clinicians of the medical facility. For example, the mapping may include weighting the real risk scores by the corresponding required risk scores, e.g., through multiplication. The new annotations are thus used to properly weight the importance of data samples in time relative to the adverse medical event. Table 1 shows an example of the initial annotations used for training the predictive algorithm in block S311, and the new annotations for re-training or fine-tuning the predictive algorithm, as discussed below, obtained by the mapping in block S314:

TABLE 1

| Time | −11 | −10 | −9 | −8 | −7 | −6 | −5 | −4 | −3 | −2 | −1 | 0 |
|---|---|---|---|---|---|---|---|---|---|---|---|---|
| Initial | 0 | 0 | 0 | 0 | 0 | 0 | 0 | 0 | 0 | 0 | 0 | 1 |
| Anno- | | | | | | | | | | | | |
| tation | | | | | | | | | | | | |
| New | 0 | 0 | 0 | 0 | 0 | 0 | 0 | 0 | 0 | .6 | .8 | 1 |
| Anno- | | | | | | | | | | | | |
| tation | | | | | | | | | | | | |

Referring to Table 1, the adverse medical event occurs at time 0. Each of the initial annotation and the new annotation is “1” at time 0, indicating the time of occurrence of the adverse medical event. The initial annotations are “0” at each of the one hour time intervals leading up to the adverse medical event, from 11 hours prior to 1 hour prior. This indicates that the initial annotations were set to “0” for these times, or that there were no initial annotations for these times. That is, the initial annotations would zero out all data samples except the data sample at time 0. The new annotation likewise is “0” at each of the one hour time intervals leading up to two hours before the adverse medical event, which is the desired notification time. However, further to the weighting, for example, performed in block S314, the new annotation is “0.6” two hours before the adverse medical event, and “0.8” one hour before the adverse medical event. That is, the new annotations would zero out data samples earlier than two hours before the adverse medical event, and additionally weight data samples at 2 and 1 hour time intervals relative to the adverse medical event. The new annotations correspond to trace 451 in FIG. 4B, which shows the required risk scores beginning to increase at two hour before the adverse medical event.

In block S315, the predictive algorithm is fine-tuned (retrained), e.g., by the AI engine (e.g., AI engine 225), using the time-series of new annotations for predicting the adverse medical event at the desired notification time. For example, the fine-tuning may be performed by creating a resampled version of the training dataset according to the new annotations. Given a loss function L(y, f(x)) that calculates the loss incurred by predicting the output f(x) of the predictive algorithm when the true label is y, the predictive algorithm would be trained to minimize the weighted loss\sum{i=1}ⁿwᵢL(yᵢ, f(xᵢ)), where w₁, w₂, . . . , wₙ are the weighted annotations for each of n samples (x₁, y₁), (x₂, y₂), . . . , (xₙ, yₙ). The fine-tuned predictive algorithm predicts the same desired notification time more precisely, in that the possibility of early prediction is eliminated. Also, the fine-tuned predictive algorithm is better calibrated to the trade-off between early notification of an adverse medical event and a false positive rate. Alternatively, a new predictive algorithm may be created, e.g., by the AI engine, based on the time-series of new annotations to accomplish the same results.

Referring again to FIG. 4A, a real cut-off line 442 extends horizontally from the vertical axis indicating a predetermined value of the real risk scores below which notification of the adverse medical event is prevented. FIG. 4B similarly shows a required cut-off line 452, which indicates the cut-off optimized for a value of the required risks scores. Thus, in an embodiment, the required cut-off line 452, together with the new annotations from the required risk scores, may be used to prevent early notification, prior to the desired notification time. The required cut-off line 452 is the threshold that may be used to activate an alert (e.g., when the required risk score exceeds the required cut-off line 452, an audible and/or visual alarm is raised). Placement of the cut-off lines 442 and 452 presents a trade-off between sensitivity and positive predictive value. For example, increasing the required cut-off line 452 will tend to decrease false positives at the expense of increasing false negatives. The real cut-off line 442 is optimized to provide the optimized required cut-off line 452 using known techniques for optimizing such trade-off between sensitivity and positive predictive value, as would be apparent to one skilled in the art. By applying the predictive algorithm to validation data, a cut-off can be selected that optimizes a particular trade-off.

In block S316, at least one of the subjects in the medical facility is monitored by applying the fine-tuned predictive algorithm to indicate the desired notification time. Data regarding each subject admitted to the medical facility may be collected and stored. Such data may include vital signs, current symptoms, and relevant medical history of the subject, for example. Based on this data, it may be determined that the subject is likely to experience an adverse medical event. A predictive algorithm corresponding to the adverse medical event may then be applied in order to provide an alert at the desired notification time prior to occurrence of the adverse medical event, according to embodiments herein. Notably, data indicating actual occurrence of the adverse medical event in the subject may be annotated, and the annotation may be used as an initial annotation for further training the predictive algorithm.

The alert at the desired notification time may include providing a visual and/or audible alarm (e.g., a tone and accompanying flashing light) at a nurses' station or other monitoring location in connection with the medical facility, or within the affected patient's room, indicating that the adverse medical event is predicted to occur in the desired notification time so that the staff is prepared. In addition, decisions may be made regarding patient management in the medical facility based on the fine-tuned predictive algorithm and/or the monitoring using the fine-tuned predictive algorithm. For example, staff and/or resource availability (e.g., number of beds, patient monitors, food service, etc.) may be adjusted to compensate for the desired notification time being greater or less than the initial notification time and for the number of patients affected by the desired notification time. For example, additional staff may need to be in place to accommodate an earlier desired notification time to comply with internal policies. Likewise, staff may be adjusted to compensate for an especially large number of patients who will be subjected to alerts corresponding to the desired notification time. Also, the medical facility may create or revise its policies regarding patient care, staffing and resource availability based on the fine-tuned predictive algorithm and/or the monitoring. Instructions regarding the adjustment of staff and resource availability may be automatically determined by a computer, and displayed or printed out in hard copy form via an interface.

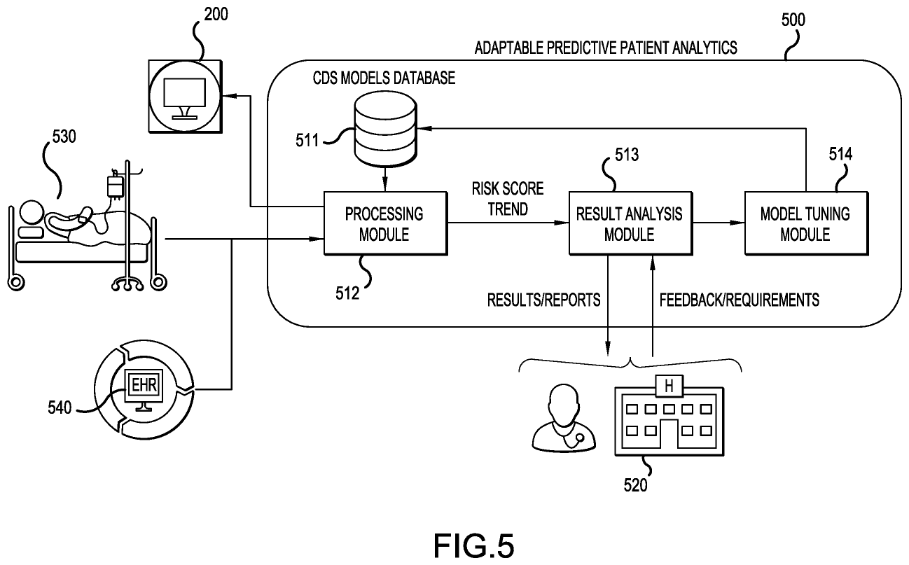

FIG. 5 is a simplified block diagram of an artificial intelligence (AI) engine for developing adaptable predictive analytics for subjects in a medical facility, according to a representative embodiment. The AI engine comprises software instructions accessible by the computer (e.g., computer 210), for implementing and automatically training the predictive algorithm, as discussed above.

Referring to FIG. 5, AI engine 500 includes a models database 511, a processing module 512, a results analysis module 513, and a model tuning module 514. The AI engine 500 interfaces with the computer system 200, as discussed above. The AI engine 500 may also receive data input from monitoring equipment attached to a subject 530 (e.g., patient) in a medical facility (e.g., medical facility 520) and an electronic health record (EHR) server 540.

The models database 511 stores a predictive algorithm or other predictive model. The predictive algorithm is developed specific to predicting a certain type of adverse medical event experienced by subjects in a medical facility, such as HI, stroke or atrial fibrillation, for example. In particular, the predictive algorithm is designed to predict timing of a desired notification time prior to the expected occurrence of the adverse medical event, so that staff of the medical facility may be notified a predetermined time before occurrence of the anticipated adverse medical event. The predictive algorithm is trained to initially provide the predicted desired notification time based on initial annotations of data collected from subjects in the medical facility 520, including from the subject 530, and optionally from other similar medical facilities. The predictive algorithm may be obtained or developed from training data collected at the medical facility.

The processing module 512 is configured to receive the predictive algorithm and associated data from the models database 511, and data from various data sources, including the monitoring equipment attached to the subject 530 and the EHR server 540, for example. The data may include laboratory measurements, vital signs, physiological waveforms, such as ECG, demographics and patient history, for example. The processing module 512 determines real risk scores using the predictive algorithm based on at least the predictive algorithm, where the real risk scores indicate actual probabilities of the adverse medical event occurring at the desired notification time before the occurrence of the adverse medical event. The data from the monitoring equipment attached to the subject 530 and the EHR server 540 may be used to train the predictive algorithm. The processing module 512 also creates required risk scores based on a real risk score trend of the real risk scores. The required risk scores indicate modified probabilities of the adverse medical event occurring at the desired notification time, removing the chances of early notification (i.e., before the desired notification time).

The required risk scores are provided by the processing module 512 to the results analysis module 513. The results analysis module 513 is configured to create and output detailed result reports for review by the clinicians at the medical facility 520. The results analysis module 513 may also receive feedback from the medical facility 520, including revised and/or additional requirements and guidance on the desired notification window, for example. The feedback may be provided manually by the clinicians via a computer system (e.g., computer system 200) at or accessible by the medical facility 520, or may be determined automatically by the computer system.

Based on the required risk scores and the feedback, the results analysis module 513 creates a set of new annotations that are provided to the model tuning module 514. For example, the results analysis module 513 may map the initial annotations to the new annotations that minimize differences between the required risk scores and the real risk scores. For example, the mapping may include weighting the real risk scores by the corresponding required risk scores, e.g., through multiplication. The model tuning module 514 is configured to fine-tune or retrain the predictive algorithm using the new annotations to more precisely predict the desired notification time for the adverse medical event, without the risk of predicting early notification times. The fine-tuned version of the predictive algorithm is stored in the models database 511 for implementation by the processing module 512. The models database 511 continues to store the original predictive algorithm as well. The AI engines 500 continues to apply and fine-tune the predictive algorithm, accordingly.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although developing adaptable predictive analytics has been described with reference to several exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Although developing adaptable predictive analytics has been described with reference to particular means, materials and embodiments, developing adaptable predictive analytics is not intended to be limited to the particulars disclosed; rather developing adaptable predictive analytics extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of developing adaptable predictive analytics for subjects in a medical facility, the method comprising:
training a predictive algorithm for predicting an adverse medical event at a desired notification time before occurrence of the adverse medical event using initial annotations indicating times for diagnosis of the adverse medical event;
determining real risk scores over time using the predictive algorithm applied to the subjects in the medical facility, and identifying a corresponding trend of the real risk scores, wherein the real risk scores indicate actual probabilities of the adverse medical event occurring at predetermined times before the occurrence of the adverse medical event;
creating required risk scores over time based on the real risk score trend, wherein the required risk scores indicate modified probabilities of the adverse medical event occurring at the predetermined times before the occurrence of the adverse medical event;
mapping the initial annotations to a time-series of new annotations that minimizes differences between the required risk scores and the real risk scores;
fine-tuning the predictive algorithm for predicting the adverse medical event at the desired notification time using the time-series of new annotations; and
monitoring at least one of the subjects in the medical facility by applying the fine-tuned predictive algorithm to indicate the desired notification time.

2. The method of claim 1, wherein mapping the initial annotations to the time-series of new annotations comprises weighting each of the real risk scores by the required risk scores, respectively.

3. The method of claim 1, wherein a trend of the required risk scores over time has a curve that is substantially the same as a curve of the real risk score trend.

4. The method of claim 1, further comprising:
weighting the new annotations and determining a cut-off of the required risk scores in order to prevent early prediction of the adverse medical event before the desired notification time.

5. The method of claim 1, further comprising adjusting patient management based on the fine-tuned predictive algorithm and/or the monitoring.

6. The method of claim 5, wherein adjusting the patient management comprises adjusting staff and/or resource availability to compensate for the desired notification time being greater or less than the initial notification time.

7. The method of claim 5, wherein adjusting the patient management comprises adjusting staff to compensate for a number of patients the desired notification time being greater or less than the initial notification time.

8. The method of claim 1, further comprising adjusting a policy of the medical facility based on the fine-tuned predictive algorithm and/or the monitoring.

9. The method of claim 1, wherein the predictive algorithm comprises a recurrent neural network (RNN)-based model.

10. The method of claim 1, wherein the adverse medical event comprises one of hemodynamics instability (HI), atrial fibrillation, acute kidney injury (AKI), pressure injury (PI), respiratory distress, acute lung injury or risk of infection.

11. The method of claim 1, further comprising:
providing a real cut-off indicating a predetermined value of the real risk scores;
optimizing the real cut-off to provide a required cut-off indicating a value of the required risk scores; and
preventing an alert or notification of the adverse medical event at values of the required risk scores below the required cut-off.

12. A system for developing adaptable predictive analytics for subjects in a medical facility, the system comprising:
an interface for receiving initial annotations indicating times for diagnosis of an adverse medical event in subjects of the medical facility;
a processor in communication with the interface; and
a memory that stores instructions that, when executed by the processor, causes the processor to perform a method comprising:
training a predictive algorithm for predicting the adverse medical event at a desired notification time before occurrence of the adverse medical event using the initial annotations;
determining real risk scores over time using the predictive algorithm applied to the subjects in the medical facility, and identifying a corresponding trend of the real risk scores, wherein the real risk scores indicate actual probabilities of the adverse medical event occurring at predetermined times before the occurrence of the adverse medical event;
creating required risk scores over time based on the real risk score trend, wherein the required risk scores indicate modified probabilities of the adverse medical event occurring at the predetermined times before the occurrence of the adverse medical event;
mapping the initial annotations to a time-series of new annotations that minimizes differences between the required risk scores and the real risk scores;
fine-tuning the predictive algorithm for predicting the adverse medical event at the desired notification time using the time-series of new annotations; and
monitoring at least one of the subjects in the medical facility by applying the fine-tuned predictive algorithm to indicate the desired notification time.

13. The system of claim 12, wherein mapping the initial annotations to the time-series of new annotations comprises weighting each of the real risk scores by the required risk scores, respectively.

14. The system of claim 12, wherein a trend of the required risk scores over time has a curve that is substantially the same as a curve of the real risk score trend.

15. The system of claim 12, wherein the instructions further cause the processor to perform weighting of the new annotations and determining a cut-off of the required risk scores in order to prevent early prediction of the adverse medical event before the desired notification time.

16. The system of claim 12, further comprising:
a display for displaying the desired notification time and/or a graph for tracking the desired notification time with respect to the subject.

17. The system of claim 16, wherein patient management is adjusted based on at least one of the displayed graph, the fine-tuned predictive algorithm or the monitoring.

18. The system of claim 12, wherein the predictive algorithm comprises a recurrent neural network (RNN)-based model.

19. A non-transitory computer readable medium that stores instructions that, when executed by a computer processor, performs a method for developing adaptable predictive analytics for subjects in a medical facility by executing steps comprising:
receiving initial annotations indicating times for diagnosis of an adverse medical event in subjects of the medical facility;
training a predictive algorithm for predicting the adverse medical event at a desired notification time before occurrence of the adverse medical event using the initial annotations;
determining real risk scores over time using the predictive algorithm applied to the subjects in the medical facility, and identifying a corresponding trend of the real risk scores, wherein the real risk scores indicate actual probabilities of the adverse medical event occurring at predetermined times before the occurrence of the adverse medical event;
creating required risk scores over time based on the real risk score trend, wherein the required risk scores indicate modified probabilities of the adverse medical event occurring at the predetermined times before the occurrence of the adverse medical event;
mapping the initial annotations to a time-series of new annotations that minimizes differences between the required risk scores and the real risk scores; and
fine-tuning the predictive algorithm for predicting the adverse medical event at the desired notification time using the time-series of new annotations, and for monitoring at least one of the subjects in the medical facility by applying the fine-tuned predictive algorithm to indicate the desired notification time.

20. The computer readable medium of claim 1, wherein mapping the initial annotations to the time-series of new annotations comprises weighting each of the real risk scores by the required risk scores, respectively.
